(19) United States
(12) Patent Application Publication
(10) Pub. No.: US 2008/0261945 A1
(43) Pub. Date: Oct. 23, 2008

(54) Compounds Having Activity at Nk3 Receptor and Uses Thereof in Medicine

(75) Inventors: Paul William Smith, Essex (GB); Paul Adrian Wyman, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION, Philidelphia, PA (US)

(21) Appl. No.: 11/718,908

(22) PCT Filed: Nov. 10, 2005
(86) PCT No.: PCT/EP2005/012210

§ 371 Date: Jan. 25, 2008

(30) Foreign Application Priority Data
Nov. 12, 2004 (GB) 0425076.7

(51) Int. Cl. A61K 31/397; C07D 215/38; A61K 31/4709; A61P 25/00; A61K 31/47

(52) U.S. Cl. 514/210.21; 546/169; 514/314; 514/311

(57) ABSTRACT

The present invention relates to compounds of formula (I), a pharmaceutically acceptable salt, solvate or prodrug thereof:

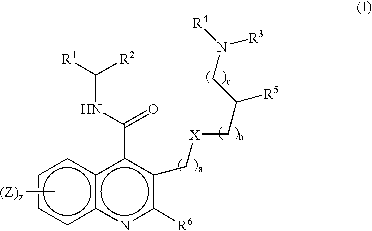

wherein R¹ is phenyl optionally substituted by 1, 2 or 3 halogen atoms which halogen atoms may be the same or different; R² is C₁₋₆alkyl, C₃₋₆cycloalkyl or acetyl; X is oxygen or sulphur; a is 1, 2 or 3; b is 0 or 1; c is 0, 1 or 2; R³ is hydrogen or C₁₋₆alkyl; R⁴ is hydrogen, C₁₋₆alkyl, haloC₁₋₆alkyl, C₁₋₄alkoxyC₁₋₆alkyl, C₃₋₆cycloalkyl or C₃₋₆cycloalkylC₁₋₆alkyl; R⁵ is hydrogen; or R⁵ and R³, together with the interconnecting atoms, form a 4, 5 or 6 membered ring; R⁶ is phenyl or thienyl, either of which is optionally substituted by 1, 2 or 3 halogen atoms, which atoms may be the same or different; and z is 0, 1 or 2; wherein when z is 1 or 2, Z is a halogen atom, and wherein when z is 2 the halogen atoms may be the same or different. Also disclosed are processes for their preparation, pharmaceutical compositions containing them and their use as medicaments particularly in treating disorders of the Central Nervous System (CNS).

The present invention relates to novel quinoline derivatives, processes for their preparation, pharmaceutical compositions containing them and their use as medicaments particularly in treating disorders of the Central Nervous System (CNS).

The mammalian peptide Neurokinin B (NKB) belongs to the Tachykinin (TK) peptide family which also includes Substance P (SP) and Neurokinin A (NKA). Pharmacological and molecular biological evidence has shown the existence of three subtypes of TK receptor (NK₁, NK₂ and NK₃) and NKB binds preferentially to the NK₃ receptor although it also recognises the other two receptors with lower affinity (Maggi et al, 1993, J. Auton. Pharmacol., 13, 23-93).

Studies examining the effects of peptidic NK₃ receptor agonists such as NKB (the endogenous agonist ligand) or senktide, have shown that activation of the NK₃ receptor has a key role in the modulation of neuronal inputs in airways, skin, spinal cord, gastrointestinal tract and within the central nervous system (Myers and Undem, 1993, J. Phisiol., 470, 665-679; Counture et al., 1993, Regul. Peptides, 46, 426-429; Mccarson and Krause, 1994, J. Neurosci., 14 (2), 712-720; Arenas et al. 1991, J. Neurosci., 11, 2332-8). Selective peptidic NK₃ receptor antagonists are known (Drapeau, 1990 Regul. Pept., 31, 125-135) and thus would be expected to reverse these agonist driven effects.

According to a first aspect, the invention provides a compound of formula (I), a pharmaceutically acceptable salt, solvate or prodrug thereof

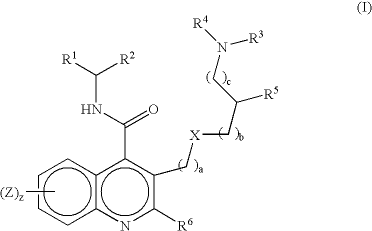

wherein
R¹ is phenyl optionally substituted by 1, 2 or 3 halogen atoms which halogen atoms may be the same or different;
R² is C₁₋₆alkyl, C₃₋₆cycloalkyl or acetyl;
X is oxygen or sulphur;
a is 1, 2 or 3;
b is 0 or 1;
c is 0, 1 or 2;
R³ is hydrogen or C₁₋₆alkyl;
R⁴ is hydrogen, C₁₋₆alkyl, haloC₁₋₆alkyl, C₁₋₄alkoxyC₁₋₆alkyl, C₃₋₆cycloalkyl or C₃₋₆cycloalkylC₁₋₆alkyl;
R⁵ is hydrogen; or R⁵ and R³, together with the interconnecting atoms, form a 4, 5 or 6 membered ring;
R⁶ is phenyl or thienyl, either of which is optionally substituted by 1, 2 or 3 halogen atoms, which atoms may be the same or different; and
z is 0, 1 or 2; wherein when z is 1 or 2, Z is a halogen atom, and wherein when z is 2 the halogen atoms may be the same or different.

Unless otherwise indicated, any alkyl group may be straight or branched and is of 1 to 6 carbon atoms, such as 1 to 4 or 1 to 3 carbon atoms.

Halo means fluoro, chloro, bromo or iodo.

In one embodiment, R¹ is optionally substituted by fluorine.

In one embodiment, R² is C₁₋₆alkyl or C₃₋₆cycloalkyl. More preferably R² is ethyl or cyclopropyl.

In one embodiment, X is oxygen.

In one embodiment, a is 1.

In one embodiment, b is 1.

In one embodiment, c is 0.

In one embodiment, R⁵ and R³, together with the interconnecting atoms, form a 4, 5 or 6 membered ring. In one embodiment, b is 1, c is 0 and R⁵ and R³, together with the interconnecting atoms, form a 5 membered ring.

In one embodiment, R⁴ is C₁₋₆alkyl.

In one embodiment, R⁶ is phenyl optionally substituted by fluorine. For example, R⁶ is unsubstituted phenyl.

In one embodiment, z is 0.

In one embodiment, the compound according to the first aspect is of formula (Ia):

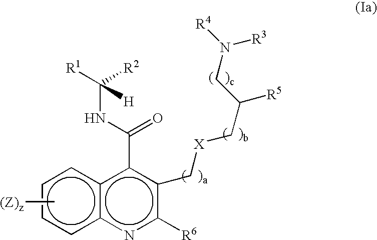

It will be appreciated that the present invention is intended to include compounds having any combination of the groups listed hereinbefore.

Example compounds of formula (I) include:
3-[({[(2S)-1-Methyl-2-pyrrolidinyl]methyl}oxy)methyl]-2-phenyl-N-[(1S)-1-phenylpropyl]-4-quinolinecarboxamide (Example 1);
N-[(1S)-2-Methyl-1-phenylpropyl]-3-[({[(2R)-1-methyl-2-pyrrolidinyl]methyl}oxy)methyl]-2-phenyl-4-quinolinecarboxamide (Example 5);
3-({[2-(Dimethylamino)ethyl]thio}methyl)-2-phenyl-N-[(1S)-1-phenylpropyl]-4-quinolinecarboxamide (Example 8);
3-({[2-(Diethylamino)ethyl]thio}methyl)-N-[(1S)-2-methyl-1-phenylpropyl]-2-phenyl-4-quinolinecarboxamide (Example 11);
2-Phenyl-N-[(1S)-1-phenylpropyl]-3-({[(2S)-2-pyrrolidinylmethyl]oxy}methyl)-4-quinolinecarboxamide (Example 12);
N-[(1S)-1-Phenylpropyl]-3-({[(2S)-2-pyrrolidinylmethyl]oxy}methyl)-2-(2-thienyl)-4-quinolinecarboxamide (Example 14);
N-[(1S)-1-Phenylpropyl]-3-({[(2S)-2-pyrrolidinylmethyl]oxy}methyl)-2-(3-thienyl)-4-quinolinecarboxamide (Example 15);
2-Phenyl-N-[(1S)-1-phenylpropyl]-3-{[(3S)-3-pyrrolidinyloxy]methyl}-4-quinolinecarboxamide (Example 16);
2-Phenyl-N-[(1S)-1-phenylpropyl]-3-{[(3R)-3-pyrrolidinyloxy]methyl}-4-quinolinecarboxamide (Example 17);
2-Phenyl-N-[(1S)-1-phenylpropyl]-3-[(4-piperidinyloxy)methyl]-4-quinolinecarboxamide (Example 19);
2-(3-Fluorophenyl)-N-[(1S)-2-methyl-1-phenylpropyl]-3-{[(3S)-3-pyrrolidinyloxy]methyl}-4-quinolinecarboxamide (Example 20);
N—[(S)-Cyclopropyl(3-fluorophenyl)methyl]-3-({[(2S)-2-pyrrolidinylmethyl]oxy}methyl)-2-(2-thienyl)-4-quinolinecarboxamide (Example 21);
3-[({[(2S)-1-Methyl-2-pyrrolidinyl]methyl}oxy)methyl]-N-[(1S)-1-phenylpropyl]-2-(2-thienyl)-4-quinolinecarboxamide (Example 26);
N—[(S)-Cyclopropyl(3-fluorophenyl)methyl]-3-[({[(2S)-1-methyl-2-pyrrolidinyl]methyl}oxy)methyl]-2-(2-thienyl)-4-quinolinecarboxamide (Example 31);
3-[({[(2S)-1-(Cyclopropylmethyl)-2-pyrrolidinyl]methyl}oxy)methyl]-2-phenyl-N-[(1S)-1-phenylpropyl]-4-quinolinecarboxamide (Example 32);
3-[({[(2S)-1-(1-Methylethyl)-2-pyrrolidinyl]methyl}oxy)methyl]-2-phenyl-N-[(1S)-1-phenylpropyl]-4-quinolinecarboxamide (Example 33);
3-{[(1-Ethyl-4-piperidinyl)oxy]methyl}-2-phenyl-N-[(1S)-1-phenylpropyl]-4-quinolinecarboxamide (Example 34);
3-({[1-(Cyclopropylmethyl)-4-piperidinyl]oxy}methyl)-2-phenyl-N-[(1S)-1-phenylpropyl]-4-quinolinecarboxamide (example 35);
3-({[1-(1-Methylethyl)-4-piperidinyl]oxy}methyl)-2-phenyl-N-[(1S)-1-phenylpropyl]-4-quinolinecarboxamide (Example 36);
3-{[(1-Cyclopentyl-4-piperidinyl)oxy]methyl}-2-phenyl-N-[(1S)-1-phenylpropyl]-4-quinolinecarboxamide (Example 37);
3-{[({(2S)-1-[2-(Methyloxy)ethyl]-2-pyrrolidinyl}methyl)oxy]methyl}-2-phenyl-N-[(1S)-1-phenylpropyl]-4-quinolinecarboxamide (Example 41);
3-[({[(2S)-1-(2-Fluoroethyl)-2-pyrrolidinyl]methyl}oxy)methyl]-2-phenyl-N-[(1S)-1-phenylpropyl]-4-quinolinecarboxamide (Example 42); and
3-[({1-[2-(Methyloxy)ethyl]-4-piperidinyl}oxy)methyl]-2-phenyl-N-[(1S)-1-phenylpropyl]-4-quinolinecarboxamide (Example 43)
   and pharmaceutically acceptable salts, solvates and prodrugs thereof.

In one embodiment, the compound is 3-[({[(2S)-1-Methyl-2-pyrrolidinyl]methyl}oxy)methyl]-2-phenyl-N-[(1S)-1-phenylpropyl]-4-quinolinecarboxamide.

It will be understood that, where appropriate, the embodiments described for the first aspect extend to further aspects described hereafter.

For the avoidance of doubt, unless otherwise indicated, the term substituted means substituted by one or more defined groups. In the case where groups may be selected from a number of alternative groups, the selected groups may be the same or different.

Suitable pharmaceutically acceptable salts of the compounds of formula (I) include mono- or di-basic salts with the appropriate acid for example organic carboxylic acids such as acetic, lactic, tartaric, malic, and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids and the like. Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of the invention may act as prodrugs of other compounds of the invention. All protected derivatives and prodrugs of compounds of the invention are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. In one embodiment, the prodrug for compounds of the invention include: amides, carbamates and sulfonamides.

Hereinafter, compounds, their pharmaceutically acceptable salts, their solvates and prodrugs, defined in any aspect of the invention (except Intermediate compounds in chemical processes) are referred to as “compounds of the invention”.

Compounds of the invention may exist in the form of optical isomers, e.g. diastereoisomers and mixtures of isomers in all ratios, e.g. racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses.

Since the compounds of the invention are intended for use in pharmaceutical compositions it will readily be understood that in one embodiment, they are each provided in substantially pure form, for example at least 60% pure, or at least 75% pure or at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and in one embodiment from 10 to 59% of a compound of the invention.

Compounds of the invention may be prepared according to the following reaction schemes. In the following reaction schemes and hereafter, unless otherwise stated R¹ to R⁶, Z, z, a, b and c are as defined in the first aspect. These processes form further aspects of the invention.

Throughout the specification, general formulae are designated by Roman numerals (I), (II), (III), (IV) etc. Subsets of these general formulae are defined as (Ia), (Ib), (Ic) etc. . . . (Iva), (Ivb), (Ivc) etc.

Compounds of formula (I) may be prepared according to reaction scheme 1 from compounds of formula (II) by reaction with compounds of formula (III) using amide coupling reagents. Suitable amide coupling reagents are HATU (O-7-azabenzotriazol-1-yl)-N,N,N′,N′-tetramethyluronium hexafluorophosphate) or a combination of EDC (1-(3-dimethylaminopropyl) 3-ethylcarbodiimide hydrochloride) and HOBt (1-hydroxybenzotriazole hydrate). In one embodiment, the reaction is carried out in the presence of a suitable base such as triethylamine or diisopropylethylamine in a suitable solvent such as DMF.

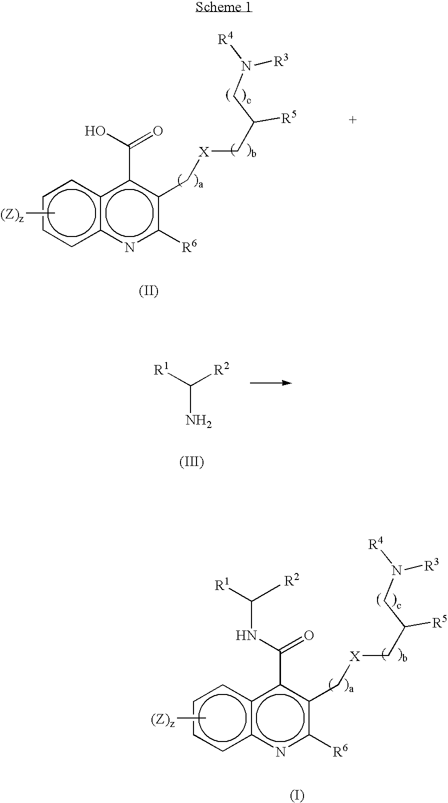

Compounds of formula (IIa), i.e. compounds of formula (II) where a is 1, may be prepared in two steps from compounds of formula (IV) according to reaction scheme 2. In the first step (V) is reacted with a suitable base (such as sodium hydride) followed by addition of the bromide (IV). The second step is hydrolysis of the methyl ester to the carboxylic acid (Iia). Suitable reaction conditions for the hydrolysis step comprise treatment with lithium hydroxide at elevated temperature, followed by acidifying with mineral acid.

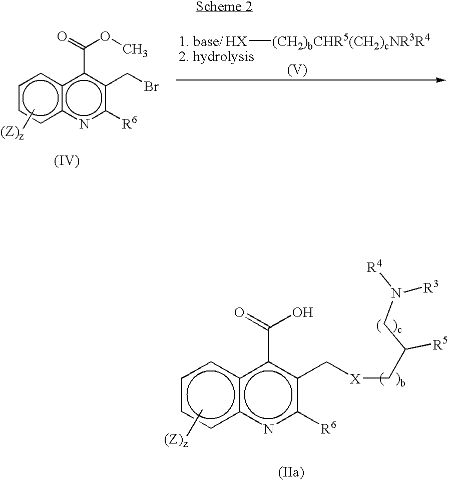

Compounds of formula (IV) may be prepared in two steps from compounds of formula (VI) according to reaction scheme 3. Compounds of formula (VI) are firstly converted to the methyl ester using one of variety of conditions. Suitable conditions comprise treatment with oxalyl chloride in a suitable solvent such as dichloromethane at room temperature catalysed by dimethyl formamide to form the acid chloride in situ, followed by treatment with methanol. Compounds of formula (IV) are then prepared by bromination. Suitable reaction conditions are treatment with N-bromosuccinimide and benzoyl peroxide in a suitable solvent (such as dimethyl carbonate) at elevated temperature.

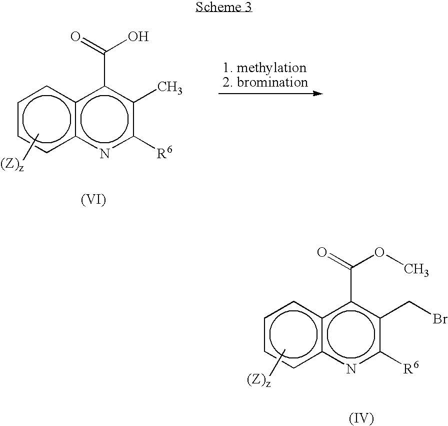

Compounds of formula (VI) may be prepared by treating compounds of formula (VII) with compounds of formula (VIII) according to reaction scheme 4. Suitable reaction conditions comprise adding concentrated hydrochloric acid to a mixture of (VII) and (VIII) in acetic acid at elevated temperatures (about 75 degC.), followed by heating under reflux. Alternative reaction conditions comprise heating a mixture of (VII) and (VIII) together with potassium hydroxide in ethanol at 80 degC. (J. Med. Chem., 1997, 40, 1794-1807).

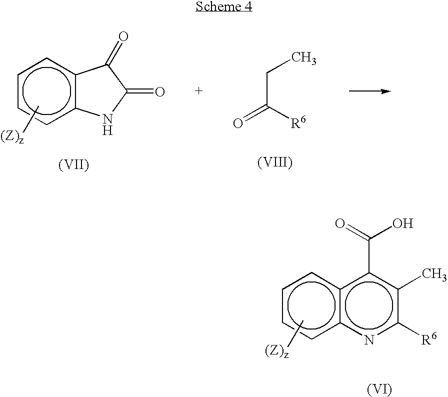

Compounds of formula (VII) are either commercially available from Sigma-Aldrich Chemicals or can be prepared using procedures described in Synthesis, 2003, 13 2047-52 or in J. Heterocyclic Chem., 1965, 2(4), 459-62.

Compounds of formula (VIII) are either commercially available from Lancaster Synthesis or can be prepared using procedures described in J. Org. Chem., 1990, 55(11), 3565-8.

Compounds of formula (IIIa) may be prepared according to reaction scheme 5 from compounds of formula (IX) by reaction with periodic acid in the presence of a suitable base such as methylamine.

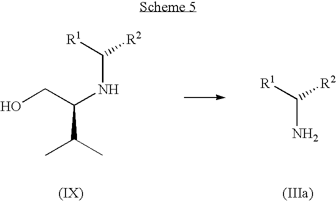

Compounds of formula (IX) may be prepared according to reaction scheme 6 from compounds of formula (X) by reaction with R²—Li (generated in situ from R²Br and tert-butyl lithium).

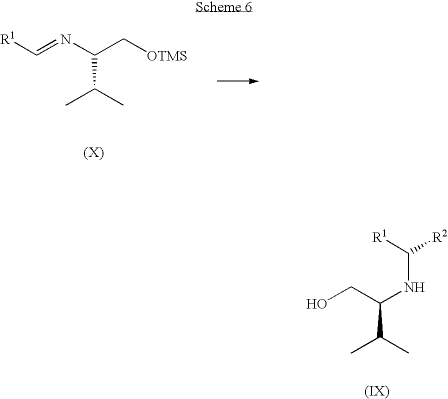

Compounds of formula (X) may be prepared according to reaction scheme 7 from commercially available benzaldehydes (XI) by reaction with valinol followed by protection of the alcohol functionality as its trimethylsilyl ether.

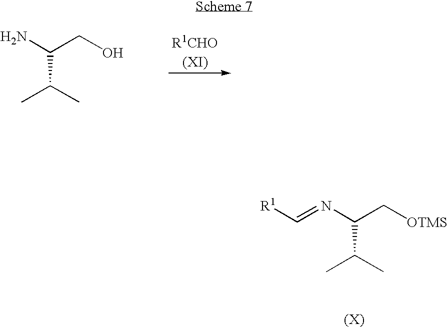

Compounds of formula (Ib), i.e. compounds of formula (I) where R⁴ is hydrogen, may be prepared according to reaction scheme 8 by reacting compounds of formula (XII) where Prot is a suitable protecting group under suitable deprotecting conditions. When Prot is tert-butyloxycarbonyl typical reaction conditions comprise treatment with trifluoroacetic acid at room temperature.

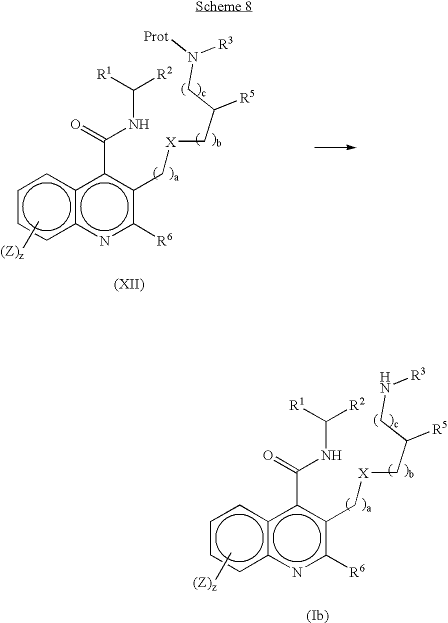

Compounds of formula (XII) may be prepared according to reaction scheme 9 by reacting compounds of formula (XIV) under basic conditions (typically sodium hydride or potassium tert-butoxide) with compounds of formula (IV) to form compounds of formula (XIII). Compounds of formula (XIII) may be converted to compounds of formula (XII) by methods similar to those described for reaction schemes 1 and 2.

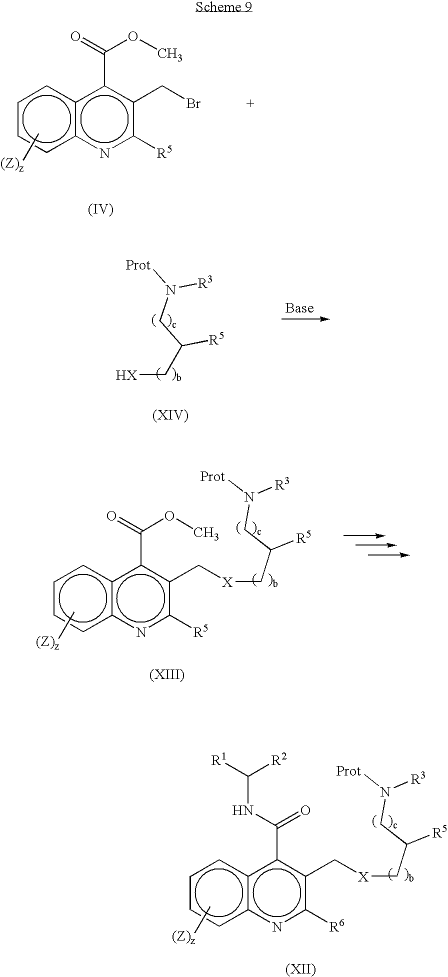

Compounds of formula (I) where R⁴ is C₁₋₆alkyl, C₃₋₆cycloalkyl or C₃₋₆cycloalkylC₁₋₆alkyl, may be prepared by reacting compounds of formula (Ib) (see scheme 8) with an appropriate aldehyde or ketone in the presences of a reducing agent typically sodium triacetoxyborohydride. When R⁴ is methyl the appropriate aldehyde is formaldehyde, when R⁴ is cyclopropylmethyl, the appropriate aldehyde is cyclopropylaldehyde.

Compounds of formula (I) where R⁴ is C₁₋₄alkoxyC₁₋₆alkyl or haloC₁₋₆alkyl may be prepared by reacting compounds of formula (Ib) (see scheme 8) with the appropriate C₁₋₄alkoxyC₁₋₆alkyl halide or haloC₁₋₆alkyl halide (respectively) in the presence of a suitable base (for example sodium carbonate).

Compounds of formula (IIa), i.e. compounds of formula (II) (see scheme 2) where a is 2, may be prepared according to reaction scheme 10. Suitable reaction conditions comprise adding concentrated hydrochloric acid to a mixture of (VII) and (XV) in acetic acid at elevated temperatures (about 75 degC.), followed by heating under reflux. Alternative reaction conditions comprise heating a mixture of (VII) and (XV) together with potassium hydroxide in ethanol at 80 degC. (J. Med. Chem., 1997, 40, 1794-1807).

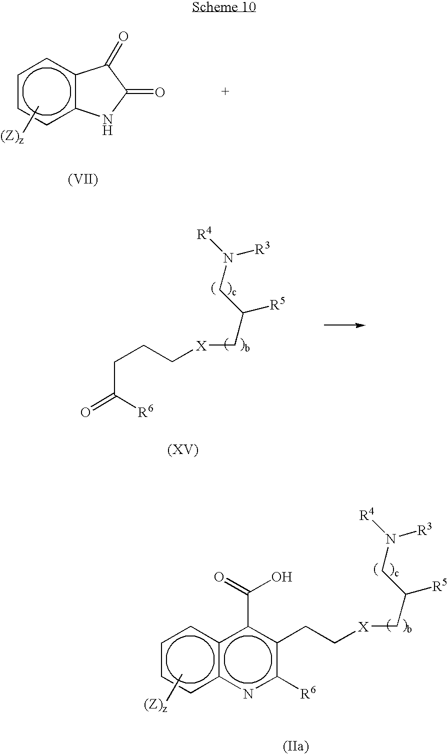

Further details for the preparation of compounds of formula (I) are found in the examples section hereinafter.

As discussed hereinabove studies examining the effects of peptidic NK₃ receptor agonists such as NKB (the endogenous agonist ligand) or senktide, have shown that activation of the NK₃ receptor has a key role in the modulation of neuronal inputs in airways, skin, spinal cord, gastrointestinal tract and within the central nervous system.

Therefore, according to a further aspect, the invention provides a compound of the invention for use as a medicament, such as a human medicament.

According to a further aspect the invention provides the use of a compound of the invention in the manufacture of a medicament for treating or preventing a disease or condition mediated by modulation of the NK₃ receptor.

In one embodiment, the diseases or conditions mediated by modulation of the NK3 receptor are CNS disorders such as depression (which term includes bipolar (manic) depression (including type I and type II), unipolar depression, single or recurrent major depressive episodes with or without psychotic features, catatonic features, melancholic features, atypical features (e.g. lethargy, over-eating/obesity, hypersomnia) or postpartum onset, seasonal affective disorder and dysthymia, depression-related anxiety, psychotic depression, and depressive disorders resulting from a general medical condition including, but not limited to, myocardial infarction, diabetes, miscarriage or abortion); anxiety disorders (including generalised anxiety disorder (GAD), social anxiety disorder (SAD), agitation, tension, social or emotional withdrawal in psychotic patients, panic disorder, and obsessive compulsive disorder); phobias (including agoraphobia and social phobia); psychosis and psychotic disorders (including schizophrenia, schizo-affective disorder, schizophreniform diseases, acute psychosis, alcohol psychosis, autism, delerium, mania (including acute mania), manic depressive psychosis, hallucination, endogenous psychosis, organic psychosyndrome, paranoid and delusional disorders, puerperal psychosis, and psychosis associated with neurodegenerative diseases such as Alzheimer's disease); post-traumatic stress disorder; attention deficit hyperactive disorder (ADHD); cognitive impairment (e.g. the treatment of impairment of cognitive functions including attention, orientation, memory (memory disorders, amnesia, amnesic disorders and age-associated memory impairment) and language function, and including cognitive impairment as a result of stroke, Alzheimer's disease, Aids-related dementia or other dementia states, as well as other acute or sub-acute conditions that may cause cognitive decline such as delirium or depression (pseudodementia states)); convulsive disorders such as epilepsy (which includes simple partial seizures, complex partial seizures, secondary generalised seizures, generalised seizures including absence seizures, myoclonic seizures, clonic seizures, tonic seizures, tonic clonic seizures and atonic seizures); psychosexual dysfunction (including inhibited sexual desire (low libido), inhibited sexual arousal or excitement, orgasm dysfunction, inhibited female orgasm and inhibited male orgasm, hypoactive sexual desire disorder (HSDD), female sexual desire disorder (FSDD), and sexual dysfunction side-effects induced by treatment with antidepressants of the SSRI-class); sleep disorders (including disturbances of circadian rhythm, dyssomnia, insomnia, sleep apnea and narcolepsy); disorders of eating behaviours (including anorexia nervosa and bulimia nervosa); neurodegenerative diseases (such as Alzheimer's disease, ALS, motor neuron disease and other motor disorders such as Parkinson's disease (including relief from locomotor deficits and/or motor disability, including slowly increasing disability in purposeful movement, tremors, bradykinesia, hyperkinesia (moderate and severe), akinesia, rigidity, disturbance of balance and co-ordination, and a disturbance of posture), dementia in Parkinson's disease, dementia in Huntington's disease, neuroleptic-induced Parkinsonism and tardive dyskinesias, neurodegeneration following stroke, cardiac arrest, pulmonary bypass, traumatic brain injury, spinal cord injury or the like, and demyelinating diseases such as multiple sclerosis and amyotrophic lateral sclerosis); withdrawal from abuse of drugs including smoking cessation or reduction in level or frequency of such activities (such as abuse of cocaine, ethanol, nicotine, benzodiazepines, alcohol, caffeine, phencyclidine and phencyclidine-like compounds, opiates such as cannabis, heroin, morphine, sedative, hypnotic, amphetamine or amphetamine-related drugs such as dextroamphetamine, methylamphetamine or a combination thereof); pain (which includes neuropathic pain (including diabetic neuropathy; sciatica; non-specific lower back pain; multiple sclerosis pain; pain associated with fibromyalgia or cancer; AIDS-related and HIV-related neuropathy; chemotherapy-induced neuropathy; neuralgia, such as post-herpetic neuralgia and trigeminal neuralgia; sympathetically maintained pain and pain resulting from physical trauma, amputation, cancer, toxins or chronic inflammatory conditions such as rheumatoid arthritis and osteoarthritis; reflex sympathetic dystrophy such as shoulder/hand syndrome), acute pain (e.g. musculoskeletal pain, post operative pain and surgical pain), inflammatory pain and chronic pain, pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia), pain associated with migraine, and non-cardiac chest pain); and certain CNS-mediated disorders (such as emesis, irritable bowel syndrome and non-ulcer dyspepsia).

Within the context of the present invention, the terms describing the indications used herein are classified in the Diagnostic and Statistical Manual of Mental Disorders, 4th Edition, published by the American Psychiatric Association (DSM-IV) and/or the International Classification of Diseases, 10th Edition (ICD-10). The various subtypes of the disorders mentioned herein are contemplated as part of the present invention. Numbers in brackets after the listed diseases below refer to the classification code in DSM-IV.

Within the context of the present invention, the term “psychotic disorder” includes:—

Schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

Compounds of formula (I) and pharmaceutically acceptable salts and solvates thereof may also be of use in the treatment of the following disorders:—

Depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90):

Anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-induced Anxiety Disorder, Separation Anxiety Disorder (309.21), Adjustment Disorders with Anxiety (309.24) and Anxiety Disorder Not Otherwise Specified (300.00):

Substance-related disorders including Substance Use Disorders such as Substance Dependence, Substance Craving and Substance Abuse; Substance-Induced Disorders such as Substance Intoxication, Substance Withdrawal, Substance-induced Delirium, Substance-induced Persisting Dementia, Substance-Induced Persisting Amnestic Disorder, Substance-Induced Psychotic Disorder, Substance-Induced Mood Disorder, Substance-induced Anxiety Disorder, Substance-induced Sexual Dysfunction, Substance-induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-induced Psychotic Disorder, Alcohol-Induced Mood Disorder, Alcohol-Induced Anxiety Disorder, Alcohol-Induced Sexual Dysfunction, Alcohol-induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-induced Mood Disorder, Hallucinogen-induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-induced Persisting Dementia, Inhalant-Induced Psychotic Disorder, Inhalant-induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide:

Sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; sleep apnea and jet-lag syndrome:

Eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; Binge Eating Disorder; and Eating Disorder Not Otherwise Specified (307.50):

Autism Spectrum Disorders including Autistic Disorder (299.00), Asperger's Disorder (299.80), Rett's Disorder (299.80), Childhood Disintegrative Disorder (299.10) and Pervasive Disorder Not Otherwise Specified (299.80, including Atypical Autism).

Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23):

Personality Disorders including the subtypes Paranoid Personality Disorder (301.0), Schizoid Personality Disorder (301.20), Schizotypal Personality Disorder (301.22), Antisocial Personality Disorder (301.7), Borderline Personality Disorder (301.83), Histrionic Personality Disorder (301.50), Narcissistic Personality Disorder (301.81), Avoidant Personality Disorder (301.82), Dependent Personality Disorder (301.6), Obsessive-Compulsive Personality Disorder (301.4) and Personality Disorder Not Otherwise Specified (301.9):

Enhancement of cognition including the treatment of cognition impairment in other diseases such as schizophrenia, bipolar disorder, depression, other psychiatric disorders and psychotic conditions associated with cognitive impairment, e.g. Alzheimer's disease: and

Sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

All of the various forms and sub-forms of the disorders mentioned herein are contemplated as part of the present invention.

In one embodiment, the diseases or conditions mediated by modulation of the NK3 receptor are depression; anxiety disorders; phobias; psychosis and psychotic disorders; post-traumatic stress disorder; attention deficit hyperactive disorder (ADHD); withdrawal from abuse of drugs including smoking cessation or reduction in level or frequency of such activities; irritable bowel syndrome; cognitive impairment; convulsive disorders; psychosexual dysfunction; sleep disorders; disorders of eating behaviours; neurodegenerative diseases; pain; emesis; irritable bowel syndrome; and non-ulcer dyspepsia.

In one embodiment, the diseases or conditions mediated by modulation of the NK3 receptor are depression; anxiety disorders; phobias; and psychosis and psychotic disorders (especially schizophrenia, schizo-affective disorder and schizophreniform diseases).

It will be appreciated that references herein to “treatment” extend to prophylaxis, prevention of recurrence and suppression or amelioration of symptoms (whether mild, moderate or severe) as well as the treatment of established conditions. The compound of the invention may be administered as the raw chemical but the active ingredient may be presented as a pharmaceutical formulation.

According to a further aspect, the invention provides a pharmaceutical composition comprising a compound of the invention, in association with one or more pharmaceutically acceptable carrier(s), diluents(s) and/or excipient(s). The carrier, diluent and/or excipient must be “acceptable” in the sense of being compatible with the other ingredients of the composition and not deletrious to the recipient thereof.

The compounds of the invention may be administered in conventional dosage forms prepared by combining a compound of the invention with standard pharmaceutical carriers or diluents according to conventional procedures well known in the art. These procedures may involve mixing, granulating and compressing or dissolving the ingredients as appropriate to the desired preparation.

The pharmaceutical compositions of the invention may be formulated for administration by any route, and include those in a form adapted for oral, topical or parenteral administration to mammals including humans.

The compositions may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatine, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatine, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilising the compound and a sterile vehicle, such as water. The compound, depending on the vehicle and concentration used, can be either suspended or dissolved in the vehicle. In preparing solutions the compound can be dissolved in water for injection and filter-sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, such as from 10-60% by weight, of the active material, depending on the method of administration. Where the compositions comprise dosage units, each unit may contain from 50-500 mg of the active ingredient. The dosage as employed for adult human treatment may range from 10 to 3000 mg per day, for instance 1500 mg per day depending on the route and frequency of administration. Such a dosage corresponds to 0.1 to 50 mg/kg per day.

It will be recognised by one of skill in the art that the optimal quantity and spacing of individual dosages of a compound of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of a compound of the invention given per day for a defined number of days, can be ascertained by those skilled in the art using conventional course of treatment determination tests.

All publications, including, but not limited to, patents and patent applications cited in this specification, are herein incorporated by reference as if each individual publication were specifically and individually indicated to be incorporated by reference herein as though fully set forth.

It will be appreciated that the invention includes the following further aspects. The embodiments described for the first aspect extend these further aspects. The disease and conditions described above extend, where appropriate, to these further aspects.

i) a compound of the invention for use in treating or preventing a disease or condition mediated by modulation of the NK3 receptor.

ii) a method of treatment or prevention of a disease or condition mediated by modulation of the NK3 receptor in a mammal comprising administering an effective amount of a compound of the invention; and

iii) a combination of a compound of the invention with an antipsychotic.

The following non-limiting examples illustrate the present invention.

### Abbreviations Used

DMF—Dimethylformamide
DMSO—Dimethylsulphoxide
DCM—Dichloromethane
EDC—1-(3-Dimethylaminopropyl) 3-ethylcarbodiimide hydrochloride
HATU—O-7-Azabenzotriazol-1-yl)-N,N,N′,N′-tetramethyluronium hexafluorophosphate
HOBt—1-hydroxybenzotriazole hydrate
HBTU—O-Benzotriazol-1-yl-N,N,N′,N′-tetramethyluronium hexafluorophosphate
THF—Tetrahydrofuran
TMS-Cl—Trimethylsilylchloride
APCI—Atmospheric Pressure Chemical Ionisation

¹H NMR spectra were recorded on a Bruker B-ACS 60 400 MHz or a Bruker DPX 400. Chemical shifts are expressed in parts per million (ppm, δ units). Coupling constants (J) are in units of hertz (Hz). Splitting patterns describe apparent multiplicities and are designated as s (singlet), d (doublet), t (triplet), q (quartet), dd (double doublet), dt (double triplet), m (multiplet), br (broad).

Mass spectra and liquid chromatography mass spectra were recorded on a Micromass MS2 Platform LC spectrometer with Agilent HP1100 Liquid Delivery system, Gilson 233 autosampler and Sedex 75 cc evaporative light scattering detector using a 4 minute run time. All mass spectra were taken under electrospray ionisation (ESI) method unless stated otherwise. Reactions were monitored by thin-layer chromatography on 0.25 mm E. Merck silica gel plates (60F-254), visualised with UV light, 5% ethanolic phosphomolybdic acid, p-anisaldehyde solution, aqueous potassium permanganate or potassium iodide/platinum chloride solution in water. Column chromatography was performed on silica gel.

### Intermediate 1: 3-Methyl-2-(3-fluorophenyl)-4-quinolinecarboxylic acid

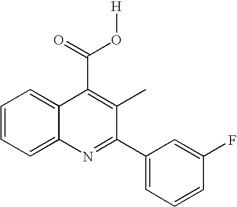

A stirred mixture of isatin (9.7 g, 66 mmole) and 3-fluoropropiophenone (10 g, 66 mmole) in acetic acid (50 ml) at 75° C. was treated with conc. HCl acid (120 ml) and then heated at reflux temperature for 20 h. The reaction mixture was allowed to cool, then poured into water (500 ml) with good stirring. After a few mins., the precipitate was filtered off, washed with water, then Et₂O, and dried. The solid was washed further by stirring in 2:1 Et₂O/EtOAc (150 ml) for 0.25 h, then filtered and dried to afford the title compound as a pale brown solid (10.3 g, 56%); ¹HNMR (400 MHz, d⁶DMSO) δ 2.39 (3H, s), 7.32-7.40 (1H, m), 7.42-7.52 (2H, m), 7.53-7.61 (1H, m), 6.67-7.73 (1H, m), 7.76-7.85 (2H, m), 8.06 (1H, d).

### Intermediate 2: 3-Methyl-2-(2-thienyl)-4-quinolinecarboxylic acid

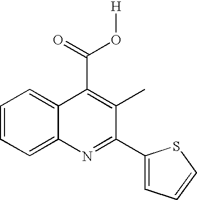

The title compound was made in a similar fashion to Intermediate 1, replacing 3-fluoropropiophenone with 1-(2-thienyl)-1-propanone; ¹HNMR (400 MHz, d⁶DMSO): δ 2.68 (3H, s), 7.22-7.27 (1H, m), 7.60-7.70 (1H, m), 7.72-7.81 (4H, m), 8.02 (1H, d).

### Intermediate 3: 3-Methyl-2-(3-thienyl)-4-quinolinecarboxylic acid

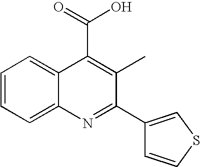

The title compound was made in a similar fashion to Intermediate 1, replacing 3-fluoropropiophenone with 1-(3-thienyl)-1-propanone; ¹HNMR (400 MHz, CD₃OD): δ 2.56 (3H, s), 7.54-7.59 (1H, m), 7.71-7.76 (1H, m), 7.79-7.84 (1H, m), 7.92-7.97 (1H, m), 8.02-8.09 (2H, m), 8.14 (1H, d).

### Intermediate 4: Methyl 3-methyl-2-(3-fluorophenyl)-4-quinolinecarboxylate (D2)

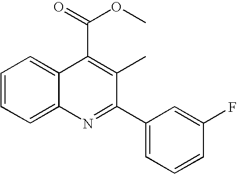

A stirred suspension of Intermediate 1 (5.7 g, 20 mmole) in DCM was treated with oxalyl chloride (6.5 g, 51 mmole), followed after a few mins by 3 drops of DMF, then the mixture was stirred at room temperature for 20 h. The solution was concentrated under vacuum and the residue dissolved in THF (100 ml), treated with MeOH (30 ml) and stirred at room temperature for 3 h. The solution was concentrated under vacuum and the residue dissolved in EtOAc and washed with 10% Na₂CO₃ solution. The organic solution was dried (MgSO₄), concentrated under vacuum and the residue purified by chromatography on silica gel eluting with 1% MeOH/DCM to afford the title product as a pale cream solid (3.32 g, 55%); ¹HNMR (400 MHz, CDCl₃) δ 2.40 (3H, s), 4.10 (3H, s), 7.12-7.20 (1H, m), 7.25-7.35 (m, 2H), 7.43-7.50 (1H, m), 7.56-7.62 (1H, m), 7.70-7.76 (2H, m), 8.14 (d, 1H).

### Intermediate 5: Methyl 3-methyl-2-(2-thienyl)-4-quinolinecarboxylate

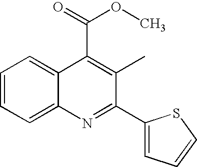

The title compound was made in a similar fashion to Intermediate 4 from Intermediate 2; ¹HNMR (400 MHz, CDCl₃): δ 2.65 (3H, s), 4.10 (3H, s), 7.12-7.19 (1H, m), 7.46-7.57 (3H, m), 7.61-7.71 (2H, m), 8.10 (1H, d).

### Intermediate 6: Methyl 3-methyl-2-(3-thienyl)-4-quinolinecarboxylate

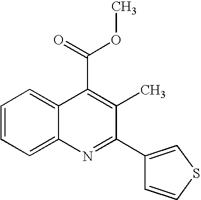

The title compound was made in a similar fashion to Intermediate 4 from Intermediate 3; ¹HNMR (400 MHz, CDCl₃): δ 2.50 (3H, s), 4.10 (3H, s), 7.40-7.48 (2H, m), 7.50-7.60 (1H, m), 7.62-7.74 (3H, m), 8.12 (1H, d).

### Intermediate 7: Methyl 3-bromomethyl-2-(3-fluorophenyl)-4-quinolinecarboxylate

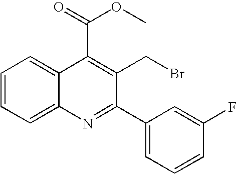

A stirred solution of Intermediate 4 (3.32 g, 11 mmole) in dimethyl carbonate (30 ml) under argon was treated with N-bromosuccinimide (2.28 g, 13 mmole) and benzoyl peroxide (0.28 g, 1.1 mmole) and then heated at 80° C. for 4 h. The mixture was concentrated under vacuum and the residue dissolved in EtOAc (75 ml), washed with water (5×25 ml), then dried (MgSO₄) and concentrated under vacuum. The residue was purified by stirring in a mixture of Et₂O (5 ml) and 60-80 petrol ether (20 ml), then filtering off the solid and drying to afford the title compound as a cream solid (3.71 g, 88%); ¹HNMR (400 MHz, CDCl₃) δ 4.17 (3H, s), 4.67 (2H, s), 7.18-7.25 (1H, m), 7.40-7.46 (1H, m), 7.48-7.52 (2H, m), 7.60-7.66 (1H, m), 7.76-7.86 (2H, m), 8.16 (1H, d).

### Intermediate 8: Methyl 3-bromomethyl-2-(2-thienyl)-4-quinolinecarboxylate

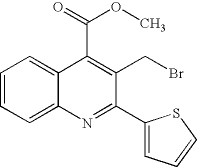

The title compound was made in a similar fashion to Intermediate 7 from Intermediate 5. ¹HNMR (400 MHz, CDCl₃): δ 4.15 (3H, s), 4.89 (2H, s), 7.17-7.22 (1H, m), 7.48-7.80 (4H, m), 7.82-7.87 (1H, m), 8.12 (1H, d).

### Intermediate 9: Methyl 3-bromomethyl-2-(3-thienyl)-4-quinolinecarboxylate

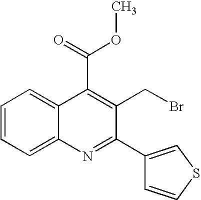

The title compound was made in a similar fashion to Intermediate 7 from Intermediate 6. ¹HNMR (400 MHz, CDCl₃): δ 4.16 (3H, s), 4.78 (2H, s), 7.47-7.52 (1H, m), 7.55-7.63 (2H, m), 7.73-7.84 (2H, m), 7.91 (1H, dd), 8.14 (1H, dd).

### Intermediate 10: Methyl 3-[({[(2S)-1-methyl-2-pyrrolidinyl]methyl}oxy)methyl]-2-phenyl-4-quinolinecarboxylate

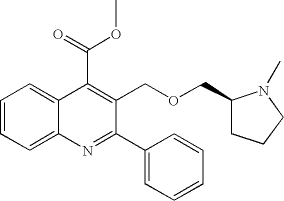

A stirred solution of (S)-1-methylpyrrolidin-2-ylmethanol (from Aldrich) (1.93 g, 15 mmole) in dry DMF (30 ml) was treated portionwise with sodium hydride (0.60 g of 60% oil dispersion, 15 mmole) and maintained at room temperature for 20 mins, then a solution of methyl 3-bromomethyl-2-phenylquinoline-4-carboxylate (J. Med. Chem. 2001, 44, 1675) (3.0 g, 8.5 mmole) in DMF (10 ml) was added and the mixture stirred at room temp. for 18 h. The reaction mixture was concentrated under vacuum and the residue treated with 10% Na₂CO₃ solution and extracted with EtOAc. The organic extract was dried (Na₂SO₄), concentrated under vacuum and the residue chromatographed on silica gel eluting with 0-4% MeOH/DCM to afford the title product as a brown gum (1.07 g, 32%); ¹H NMR (400 MHz, CDCl₃) δ1.48-1.56 (1H, m), 1.62-1.75 (m, 2H), 1.83-1.95 (m, 1H), 2.15-2.25 (m, 1H), 2.30-2.40 (m, 1H), 2.34 (s, 3H), 3.05-3.27 (m, 1H), 3.28-3.40 (m, 1H), 3.39-3.45 (m, 1H), 4.08 (s, 3H), 4.67 (s, 2H), 7.45-7.52 (m, 3H), 7.56-7.62 (m, 3H), 7.75 (t, 1H), 7.87 (t, 1H), 8.17 (d, 1H); MH⁺ 391.

The following compounds of formula (XVI) (see table 1) were prepared from methyl 3-bromomethyl-2-phenylquinoline-4-carboxylate (J. Med. Chem. 2001, 44, 1675) and the appropriate alcohol or thiol in a similar fashion to the preparation of Intermediate 10.

TABLE 1

| | |
|---|---|
| | (XVI) |
| 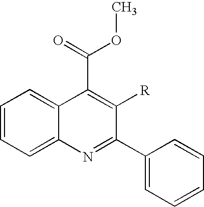 | |

| Int | R |
|---|---|
| 11 | 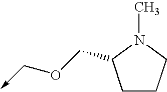 |
| 12 | 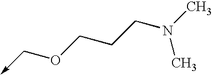 |
| 13 | 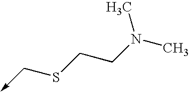 |
| 14 | 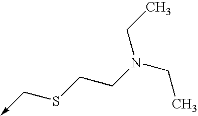 |

### Intermediate 15: 3-((S)-1-Methylpyrrolidin-2-ylmethoxymethyl)-2-phenylquinoline-4-carboxylic acid hydrochloride

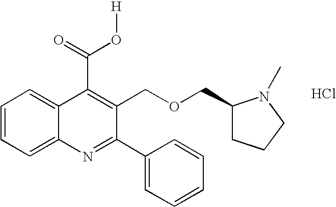

A stirred solution of Intermediate 10 (1.07 g, 2.7 mmole) in EtOH (10 ml) was treated with a solution of LiOH.H₂O (0.34 g, 8.2 mmole) in H₂O (10 ml) and heated at reflux for 8 h. The mixture was acidified with conc. HCl acid to pH1, then concentrated under vacuum followed by drying under vacuum at 45° C. for 5 h to afford the title compound plus LiCl as a red solid (1.74 g, quantitative); MH⁺ 377.

The following compounds of formula (XVII) (see table 2) were prepared in a similar fashion to the preparation of Intermediate 15, from the starting materials indicated.

TABLE 2

| | |
|---|---|
| | (XVII) |
| 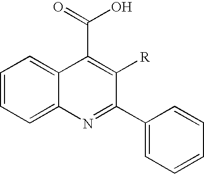 | |

| | | Starting |
|---|---|---|
| Int. | R | material |
| 16 | 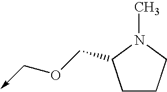 | Int 11 |
| 17 | 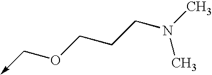 | Int 12 |
| 18 | 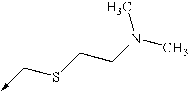 | Int 13 |
| 19 | 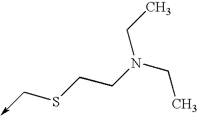 | Int 14 |

### Intermediate 20: Methyl 3-({[((2S)-1-{[(1,1-dimethylethyl)oxy]carbonyl}-2-pyrrolidinyl)methyl]oxy}methyl)-2-phenyl-4-quinolinecarboxylate

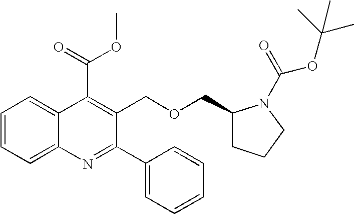

A stirred solution of methyl 3-bromomethyl-2-phenylquinoline-4-carboxylate (J. Med. Chem. 2001, 44, 1675) (1.5 g, 4.2 mmole) and (S)-1-tert-butoxycarbonyl-2-pyrrolidinemethanol (from Lancaster Synthesis) (1.0 g, 5.0 mmole) in dry THF (45 ml) at 0° C. under argon was treated with solid potassium tert-butoxide (0.53 g, 4.7 mmole), then allowed to warm to room temperature over 1 hr. The mixture was concentrated under vacuum and the residue treated with 10% Na₂CO₃ solution and extracted with EtOAc. The extract was dried (Na₂SO₄), concentrated under vacuum and the residue chromatographed on silica gel eluting with 0-5% Et₂O/DCM to afford the title compound as a white solid (1.30 g, 65%); ¹H NMR (400 MHz, CDCl₃) δ 1.30-1.55 (9H, br d), 1.70-1.95 (4H, m), 3.17-3.60 (4H, m), 3.80 & 3.95 (together 1H, 2×br s), 4.09 (3H, s), 4.58-4.72 (2H, m), 7.45-7.53 (3H, m), 7.55-7.65 (3H, m), 7.75 (1H, t), 7.85 (1H, d), 8.16 (1H, d).

The following compounds of formula (XVIII) (see Table 3) were prepared by a similar procedure to that of Intermediate 20 from the starting materials indicated.

TABLE 3

| | |
|---|---|
| | (XVIII) |
| 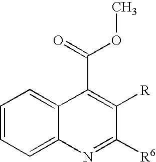 | |

| | | | Starting | |
|---|---|---|---|---|
| Int | R | R⁶ | bromide | Starting alcohol |
| 21 | 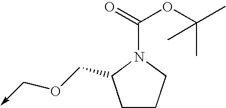 | phenyl | J. Med. Chem.2001, 44, 1675 | 1,1-dimethylethyl-(2R)-2-(hydroxymethyl)-1-pyrrolidinecarboxylate(Lancaster Synthesis) |
| 22 | 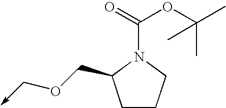 | 2-thienyl | Int 8 | 1,1-dimethylethyl-(2S)-2-(hydroxymethyl)-1-pyrrolidinecarboxylate(Lancaster Synthesis) |
| 23 | 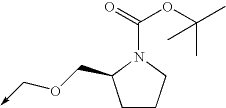 | 3-thienyl | Int 9 | 1,1-dimethylethyl-(2S)-2-(hydroxymethyl)-1-pyrrolidinecarboxylate(Lancaster Synthesis) |
| 24 | 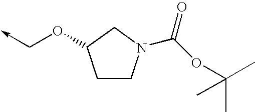 | phenyl | J. Med. Chem.2001, 44, 1675 | 1,1-dimethylethyl-(3S)-3-hydroxy-1-pyrrolidinecarboxylate(Lancaster Synthesis) |
| 25 | 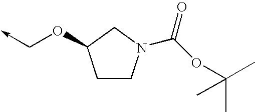 | phenyl | J. Med. Chem.2001, 44, 1675 | 1,1-dimethylethyl-(3R)-3-hydroxy-1-pyrrolidinecarboxylate(Sigma-Aldrich Chemicals) |
| 26 | 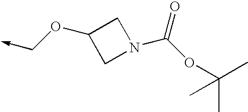 | phenyl | J. Med. Chem.2001, 44, 1675 | 1,1-dimethylethyl-3-hydroxy-1-azetidinecarboxylate(Intermediate 58) |
| 27 | 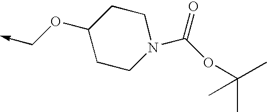 | phenyl | J. Med. Chem.2001, 44, 1675 | 1,1-dimethylethyl-4-hydroxy-1-piperidinecarboxylate(Sigma-Aldrich Chemicals) |
| 28 | 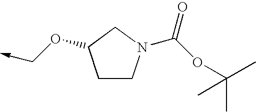 | 3-F-phenyl | Int 7 | 1,1-dimethylethyl-(3S)-3-hydroxy-1-pyrrolidinecarboxylate(Lancaster Synthesis) |

### Intermediate 29: 3-({[((2S)-1-{[(1,1-Dimethylethyl)oxy]carbonyl}-2-pyrrolidinyl)methyl]oxy}methyl)-2-phenyl-4-quinolinecarboxylic acid (D7)

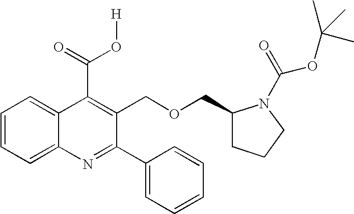

A stirred solution of Intermediate 20 (1.12 g, 2.35 mmole) in MeOH (5 ml) and THF (6 ml) was treated with a solution of LiOH (0.34 g, 14 mmole) in water (20 ml) and heated at reflux for 3 hrs, then further LiOH (0.23 g) was added and heating continued for a further 9 hrs. The solution was concentrated to approx. 20 ml volume, then acidified with excess aqueous citric acid solution and extracted with EtOAc. The extract was washed with water (×2), then dried (Na₂SO₄) and concentrated under vacuum to afford the title compound as a beige solid (1.02 g, 94%); ¹H NMR (400 MHz, CDCl₃) δ 1.42 (9H, s), 1.65-1.95 (4H, m), 3.15-3.60 (4H, m), 3.85 & 4.05 (together 1H, 2×br s), 4.60-4.85 (2H, m), 7.45-7.52 (3H, m), 7.53-7.65 (3H, m), 7.75 (1H, t), 7.95-8.10 (1H, m), 8.17 (1H, d), 8.70 (1H, vr br s).

The following compounds of formula (XIX) (see Table 4) were prepared by a similar procedure to that of Intermediate 29, from the starting materials indicated.

TABLE 4

| | | | (XIX) |
|---|---|---|---|
| 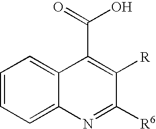 | | | |
| | | | Starting |
| Int | R | R⁶ | material |
| 30 | 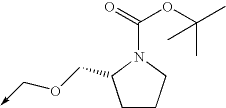 | phenyl | Int 21 |
| 31 | 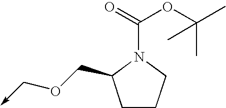 | 2-thienyl | Int 22 |
| 32 | 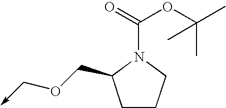 | 3-thienyl | Int 23 |
| 33 | 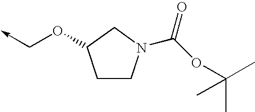 | phenyl | Int 24 |
| 34 | 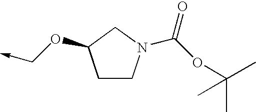 | phenyl | Int 25 |
| 35 | 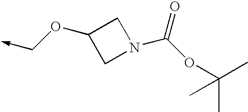 | phenyl | Int 26 |
| 36 | 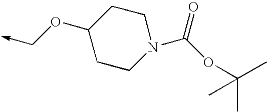 | phenyl | Int 27 |
| 37 | 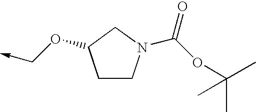 | 3-F-phenyl | Int 28 |

### Intermediate 38: 1,1-Dimethylethyl (2S)-2-[({[2-phenyl-4-({[(1S)-1-phenylpropyl]amino}carbonyl)-3-quinolinyl]methyl}oxy)methyl]-1-pyrrolidinecarboxylate

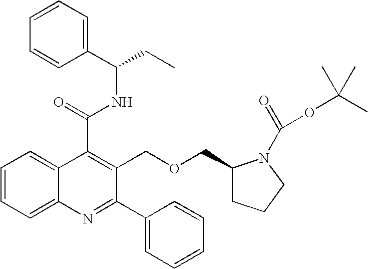

A stirred solution of Intermediate 29 (1.36 g, 2.9 mmole) in DMF (35 ml) at room temperature under argon was treated with diisopropylethylamine (1.0 ml, 5.8 mmole) and (S)-1-phenylpropylamine (0.47 g, 3.5 mmole), then HATU (O-7-azabenzotriazol-1-yl)-N,N,N′,N′-tetramethyluronium hexafluorophosphate) (1.29 g, 3.4 mmole) was added and the mixture then maintained for 4 days. The solution was concentrated under vacuum and the residue dissolved in EtOAc and washed quickly with 0.5M HCl acid followed immediately by 10% Na₂CO₃ solution. The organic solution was dried (Na₂SO₄) and concentrated under vacuum. The residue was chromatographed on silica gel eluting with 0-6% Et₂O/DCM to afford the title compound as a beige coloured solid (1.50 g, 88%); ¹H NMR (400 MHz, CDCl₃) δ 1.05 (3H, br m), 1.33 (9H, br s), 1.55-1.85 (4H, m), 1.90-2.20 (2H, m), 2.90 & 3.10-3.40 & 3.50-3.85 (together 5H, several m), 4.25-4.60 (2H, m), 5.23-5.36 (1H, m), 6.80 & 7.20-7.58 (together 9H, m), 7.60-7.66 (2H, m), 7.73 (1H, t), 7.92 (1H, br s), 8.13 (1H, d).

The following compounds of formula (XX) (see Table 5) were prepared by a similar procedure to that of Intermediate 38 from the starting materials indicated.

TABLE 5

| | |
|---|---|
| | (XX) |
| 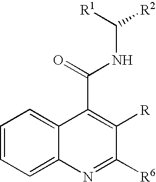 | |

| | | | | | Starting | Starting |
|---|---|---|---|---|---|---|
| Int | R¹ | R² | R⁶ | R | acid | amine |
| 39 | phenyl | ethyl | phenyl | 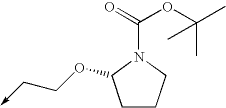 | Int 30 | (S)-1-phenyl-propylamine |
| 40 | phenyl | ethyl | 2-thienyl | 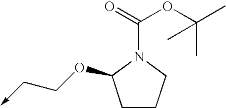 | Int 31 | (S)-1-phenyl-propylamine |
| 41 | phenyl | ethyl | 3-thienyl | 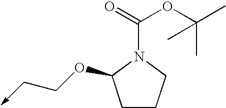 | Int 32 | (S)-1-phenyl-propylamine |
| 42 | phenyl | ethyl | phenyl | 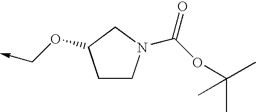 | Int 33 | (S)-1-phenyl-propylamine |
| 43 | phenyl | ethyl | phenyl | 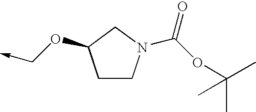 | Int 34 | (S)-1-phenyl-propylamine |
| 44 | phenyl | ethyl | phenyl | 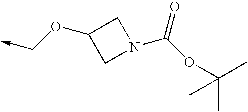 | Int 35 | (S)-1-phenyl-propylamine |
| 45 | phenyl | ethyl | phenyl | 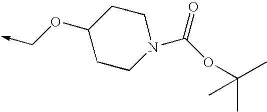 | Int 36 | (S)-1-phenyl-propylamine |
| 46 | phenyl | iso-propyl | 3-F-phenyl | 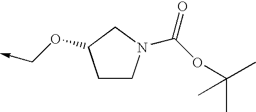 | Int 37 | (S)-2-methyl-1-phenyl-propylamine |
| 47 | 3-F-phenyl | cyclo-propyl | 2-thienyl | 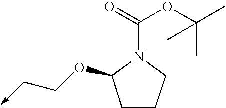 | Int 31 | Int 56 |
| 48 | 3-F-phenyl | cyclo-propyl | phenyl | 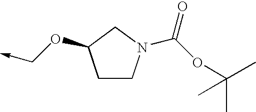 | Int 33 | Int 55 |
| 49 | 3-F-phenyl | ethyl | phenyl | 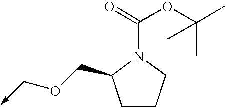 | Int 29 | (S)-1-(3-fluorophenyl)-propylamine;Example 69;WO02/083624 |

### Intermediate 50: (S)-2-(Benzylideneamino)-3-methylbutan-1-ol

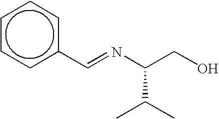

(S)-(+)-Valinol (4.16 g, 40.3 mmole) was dissolved in dichloromethane (60 ml) and magnesium sulphate (20 g) was added. The mixture was cooled to 0° C. and treated dropwise with benzaldehyde (4.28 g, 40.3 mmole). Stirring was continued at 0° C. for 2 hrs and then at ambient temperature for 18 hrs. The reaction mixture was filtered and evaporated in vacuo to afford the title compound as a white solid (6.7 g, 87%); m/z (APCI): 192.16 [M+H]⁺.

### Intermediate 51: (S)-2-[(3-Fluorobenzylidene)amino]-3-methylbutan-1-ol

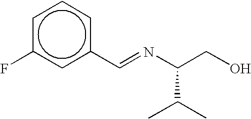

The title compound was prepared in a similar manner to Intermediate 50 using 3-fluorobenzaldehyde and was isolated as a pale brown oil (16.72 g, 99%); m/z (APCI): 210.2 [M+H]⁺.

### Intermediate 52: (2S)-3-Methyl-N-[(1E)-phenylmethylidene]-1-[(trimethylsilyl)oxy]-2-butanamine

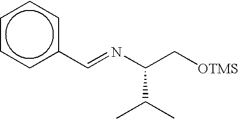

Intermediate 50 (6.7 g, 35 mmole) was dissolved in dry dichloromethane (60 ml) and treated with triethylamine (5.4 ml, 38.5 mmole) and trimethylsilyl chloride (4.9 ml, 38.5 mmole) under argon. The mixture was stirred at ambient temperature for 72 hrs, filtered and then evaporated to dryness. The residue was triturated with Et₂O and the filtrate evaporated to dryness under vacuum to afford the title compound (8.43 g, 91%) as a colourless oil; ¹HNMR (400 MHz, CDCl₃) δ 0.01 (9H, s), 0.88-0.90, (6H, m), 1.87-1.95, (1H, m), 2.92-2.97, (1H, m), 3.59-3.64, (1H, m), 3.82-3.85, (1H, m), 7.22-7.37, (3H, m), 7.68-7.73, (2H, m), 8.17, (1H, s).

### Intermediate 53: (2S)—N-[(1E)-(3-Fluorophenyl)methylidene]-3-methyl-1-[(trimethylsilyl)oxy]-2-butanamine

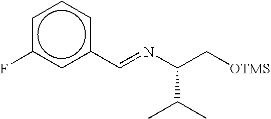

The title compound was prepared in a similar manner to Intermediate 52 using Intermediate 51 as starting material and was isolated as a pale brown oil (22.12 g, 98%); ¹HNMR (400 MHz, CDCl₃) δ 0.01 (9H, s), 0.86-0.90, (6H, m), 1.87-1.95, (1H, m), 2.94-2.98, (1H, m), 3.58-3.63, (1H, m), 3.81-3.84, (1H, m), 7.04-7.06, (1H, m), 7.32-7.35, (1H, m), 7.42-7.48, (2H, m), 8.13, (1H, s).

### Intermediate 54: (2S)—N—[(S)-cyclopropyl(phenyl)methyl]-3-methyl-1-[(trimethylsilyl)oxy]-2-butanamine

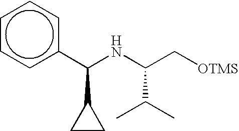

Cyclopropyl bromide (4.64 g, 38.4 mmole) was dissolved in dry Et₂O (50 ml) under argon, cooled to −78° C. and treated with tert-BuLi (45 mL of a 1.7M solution in pentane, 76.5 mmole). After 10 minutes, cooling was removed and the mixture stirred at room temperature for 1 hr. After re-cooling to −40° C., a solution of Intermediate 52 (8.43 g, 32 mmole) in dry Et₂O (40 ml) was added and stirring continued at −40° C. for 1.5 hrs. 5M HCl acid was added (50 ml) and the phases separated. The aqueous phase was washed with Et₂O (discarded) and then basified with KOH pellets to pH>10 in the presence of Et₂O. The organic phase was washed with water and brine and then evaporated to dryness under vacuum to afford the title compound as a colourless oil (6.42 g, 86%); ¹HNMR (400 MHz, CDCl₃) δ 0.13-0.15, (1H, m), 0.34-0.37, (2H, m), 0.60-0.70, (1H, m), 0.83, (3H, d, J=7 Hz), 0.91, (3H, d, J=7 Hz), 0.98-1.00, (1H, m), 1.71-1.77, (1H, m), 2.44-2.48, (1H, m), 3.00, (1H, d, J=8 Hz), 3.32 and 3.36, (1H, dd, J=5 and 11 Hz), 3.59 and 3.61, (1H, dd, J=5 and 11 Hz), 7.25-7.42, (5H, m); m/z (APCI): 234.2 [M+H]⁺.

### Intermediate 55: (2S)—N—[(S)-cyclopropyl(3-fluorophenyl)methyl]-3-methyl-1-[(trimethylsilyl)oxy]-2-butanamine

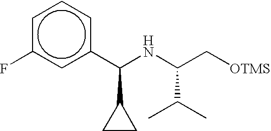

The title compound was prepared in a similar manner to Intermediate 54 using Intermediate 53 as starting material and was isolated as a brown oil (15.47 g, 91%); ¹HNMR (400 MHz, CDCl₃) δ 0.15-0.17, (1H, m), 0.35-0.38, (2H, m), 0.65-0.67, (1H, m), 0.83, (3H, d, J=7 Hz), 0.91, (3H, d, J=7 Hz), 1.00-1.03, (1H, m), 1.70-1.77, (1H, m), 2.40-2.44, (1H, m), 2.99, (1H, d, J=9 Hz), 3.36 and 3.38, (1H, dd, J=5 and 11 Hz), 3.59 and 3.62, (1H, dd, J=5 and 11 Hz), 6.94-6.97, (1H, m), 7.03-7.08 (2H, m), 7.26-7.29 (1H, m)

### Intermediate 56: (S)-1-Cyclopropyl-1-phenylmethylamine hydrochloride

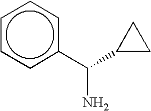

Intermediate 54 (1.67 g, 7.2 mmole) was dissolved in methanol (20 ml) and aqueous methylamine (9 ml of a 40% solution in water) added. This mixture was treated with a solution of H₅IO₆ (5.30 g, 23.3 mmole) in water (5 ml). An initial exotherm was observed (approx 50° C.). After 24 hrs at ambient temperature, some starting material was evident by TLC (NH₃/MeOH/CH₂Cl₂ 1:9:90), so the mixture was heated to reflux for 30 mins. After cooling to room temperature, a further portion of H₅IO₆ (1.8 g, 7.9 mmole) in water (5 ml) and aqueous methylamine (5 ml) were added and stirring continued for a further 18 hrs at ambient temperature. All insoluble material was removed by filtration and washed with methanol. The filtrate and washings were concentrated under vacuum and the residue partitioned between Et₂O (×5) and water. The combined organic extracts were concentrated to low volume under vacuum, treated with 5M HCl acid (10 ml) and stirred for 18 hrs at ambient temperature. After reduction to a small volume, the residue was washed with Et₂O and then basified with KOH pellets (to pH>10) in the presence of Et₂O. The phases were separated and the organic phase washed with water, saturated brine and dried (MgSO₄). The filtrate was treated with HCl (10 ml of a 1M solution in ether) and the product collected by filtration (0.972 g, 74%); ¹HNMR (400 MHz, d⁶DMSO) δ 0.36-0.38, (1H, m), 0.47-0.49, (1H, m), 0.60-0.65, (2H, m), 1.30-1.35, (1H, m), 3.54-3.58, (1H, m), 7.35-7.44, (3H, m), 7.55-7.58, (2H, m), 8.71, (3H, brs, exchangeable); [α]²⁸_{D}=+ 45.9° (c=1 in MeOH).

### Intermediate 57: (S)-1-Cyclopropyl-1-(3-fluorophenyl)methylamine hydrochloride

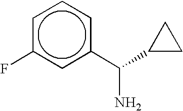

The title compound was prepared in a similar manner to Intermediate 56 using Intermediate 55 as starting material and was isolated as a cream solid (4.36 g, 72%); ¹HNMR (400 MHz, d⁶DMSO) δ 0.39-0.42, (1H, m), 0.47-0.51, (1H, m), 0.60-0.67, (2H, m), 1.29-1.35, (1H, m), 3.59-3.62, (1H, m), 7.20-7.24, (1H, m), 7.39-7.41 (1H, m), 7.45-7.51, (2H, m), 8.73, (3H, br s, exchangeable); [α]²⁵_{D}=+42.1° (c=1 in EtOH).

### Intermediate 58: 1,1-Dimethylethyl 3-hydroxy-1-azetidinecarboxylate

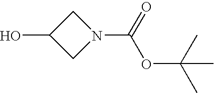

A stirred solution of 1-benzylazetidinol (J. Het. Chem. 1987, 24(1), 255-9) (0.50 g, 3.0 mmole) in MeOH (15 ml) and formic acid (1 ml) at room temperature under argon was treated with a slurry of 10% Pd—C catalyst (0.20 g) in MeOH (5 ml) and the mixture stirred well for 20 h, then filtered through a pad of Kieselguhr. The filtrate was treated with triethylamine (1 ml) and di-tert-butyl dicarbonate (0.65 g, 3.0 mmole), then stirred at room temperature for 24 h. The solution was concentrated under vacuum and the residue treated with 10% Na₂CO₃ solution and extracted with EtOAc. The extract was dried (Na₂SO₄), concentrated under vacuum and the residue chromatographed on silica gel eluting with Et₂O to afford the title compound as a white crystalline solid (0.32 g, 60%); ¹HNMR (400 MHz, CDCl₃) δ 1.43 (9H, s), 2.66 (1H, br s), 3.79 (1H, dd), 4.14 (1H, dd), 4.57 (1H, m).

### EXAMPLE 1

### 3-[({[(2S)-1-Methyl-2-pyrrolidinyl]methyl}oxy)methyl]-2-phenyl-N-[(1S)-1-phenylpropyl]-4-quinolinecarboxamide

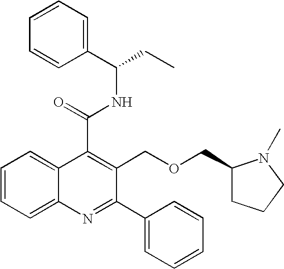

A stirred solution of Intermediate 15 (0.30 g, 0.67 mmole) in dry DMF (15 ml) was treated with HBTU (0.38 g, 1.0 mmole) and maintained at room temp. for 20 mins., then (S)-1-phenylpropylamine (from Lancaster Synthesis) (0.15 ml, 1.0 mmole) was added and the mixture stirred at room temp. for 72 h. The solution was concentrated under vacuum and the residue was dissolved in EtOAc and extracted with 1M HCl acid. The acid layer was separated, basified with solid K₂CO₃ and extracted with EtOAc. The extract was dried (Na₂SO₄), concentrated under vacuum and the residue chromatographed on silica gel eluting with 0-5% MeOH/DCM to afford the title compound as a yellow gum (46 mg, 14%). This was converted to its hydrochloride salt to afford a solid; ¹H NMR (400 MHz, CDCl₃) δ 1.03 (t, 3H), 1.35-2.20 (several m, 8H), 2.22 (s, 3H) 3.02 (br s, 2H), 3.30 (br s, 1H), 4.40-4.50 (m, 2H), 5.20 (q, 1H), 7.15 (br s, 1H), 7.28-7.50 (m, 8H), 7.54 (t, 1H), 7.63-7.69 (m, 2H), 7.73 (t, 1H), 7.90 (br s, 1H), 8.13 (d, 1H); m/z (APCI): 494.21 [M+H]⁺; Retention time 2.40 mins.

The following compounds of formula (Ic) (see table 6) were prepared by methods similar to that of Example 1 from the starting materials indicated.

TABLE 6

| | |
|---|---|
| | (Ic) |
| 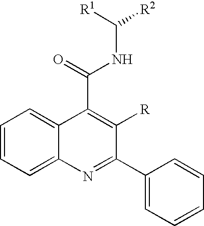 | |

| | | | | | | m/z |
|---|---|---|---|---|---|---|
| | | | | starting | starting | (retention |
| Ex | R¹ | R² | R | acid | amine | time) |
| 2 | phenyl | ethyl | 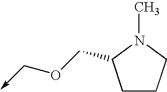 | Int 16 | (S)-1-phenyl-propylamine | 494.3(2.46mins) |
| 3 | phenyl | methyl | 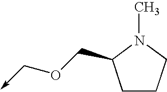 | Int 15 | (S)-1-phenyl-propylamine | 480.3(2.39mins) |
| 4 | phenyl | methyl | 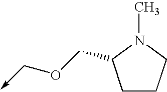 | Int 16 | (S)-1-phenyl-propylamine | 480.2(2.39mins) |
| 5 | phenyl | isopropyl | 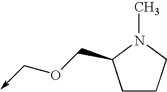 | Int 16 | (S)-2-methyl-1-phenyl-propylamine | 508.3(2.49mins) |
| 6 | 3-F-phenyl | cyclo-propyl | 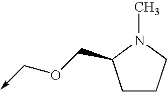 | Int 15 | Int 57 | 524.6(2.15mins) |
| 7 | phenyl | ethyl | 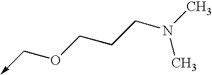 | Int 17 | (S)-1-phenyl-propylamine | 482.3(2.47mins) |
| 8 | phenyl | ethyl | 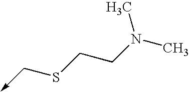 | Int 18 | (S)-1-phenyl-propylamine | 484.2(2.40mins) |
| 9 | phenyl | ethyl | 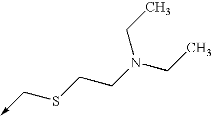 | Int 19 | (S)-1-phenyl-propylamine | 512.2(2.49mins) |
| 10 | phenyl | methyl | 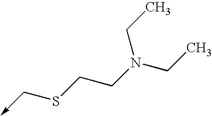 | Int 19 | (S)-1-phenyl-propyl-amine | 498.2(2.42mins) |
| 11 | phenyl | isopropyl | 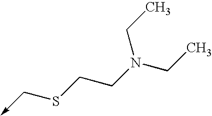 | Int 19 | (S)-2-methyl-1-phenyl-propylamine | 526.3(2.55mins) |

### EXAMPLE 12

### 2-Phenyl-N-[(1S)-1-phenylpropyl]-3-({[(2S)-2-pyrrolidinylmethyl]oxy}methyl)-4-quinolinecarboxamide

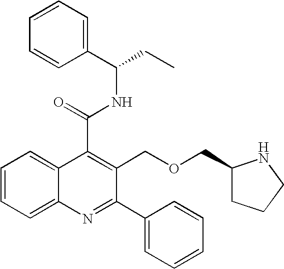

A stirred solution of Intermediate 38 (1.50 g, 2.6 mmole) in DCM (25 ml) at room temperature under argon was treated with trifluoroacetic acid (5 ml) and maintained for 3 hrs. The mixture was concentrated under vacuum and the residue dissolved in EtOAc, washed with 10% Na₂CO₃ solution, then dried (Na₂SO₄) and concentrated under vacuum to afford the title compound as a beige foam (1.25 g, 100%); ¹H NMR (400 MHz, CDCl₃) δ: 1.02 (3H, t), 1.08-1.28 (1H, m), 1.60-1.78 (3H, m), 1.90-2.00 (1H, m), 2.00-2.15 (1H, m), 2.60-3.20 (6H, m), 4.39 (2H, br s), 5.30 (1H, q), 7.00-7.10 (1H, m), 7.22-7.50 (8H, m), 7.50-7.60 (1H, m), 7.60-7.68 (2H, m), 7.70-7.80 (1H, m), 7.94 (1H, br s), 8.14 (1H, d); m/z 480.2 [M+H]⁺; Retention time 2.45 mins.

The following compounds of formula (Id) (see table 7) were prepared using methods similar to that of Example 12 from the starting materials indicated.

TABLE 7

| | |
|---|---|
| | (Id) |
| 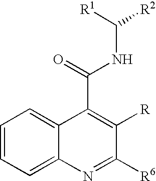 | |

| | | | | | | m/z |
|---|---|---|---|---|---|---|
| | | | | | Starting | (retention |
| Ex | R¹ | R² | R⁶ | R | material | time) |
| 13 | 3-F-phenyl | ethyl | phenyl | 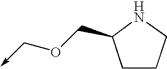 | Int 49 | 498.3(2.48mins) |
| 14 | phenyl | ethyl | 2-thienyl | 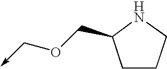 | Int 40 | 486.3(2.17mins) |
| 15 | phenyl | ethyl | 3-thienyl | 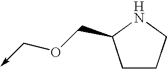 | Int 41 | 486.2(2.19mins) |
| 16 | phenyl | ethyl | phenyl | 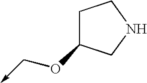 | Int 43 | 466.3(2.35mins) |
| 17 | phenyl | ethyl | phenyl | 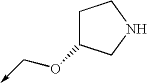 | Int 42 | 466.3(2.34mins) |
| 18 | phenyl | ethyl | phenyl | 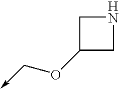 | Int 44 | 452.2(2.31mins) |
| 19 | phenyl | ethyl | phenyl | 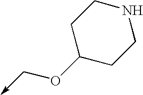 | Int 45 | 480.3(2.34mins) |
| 20 | phenyl | iso-propyl | 3-F-phenyl | 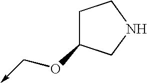 | Int 46 | 498.2(2.37mins) |
| 21 | 3-F-phenyl | cyclo-propyl | 2-thienyl | 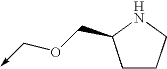 | Int 47 | 516.3(2.25mins) |
| 22 | 3-F-phenyl | cyclo-propyl | phenyl | 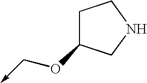 | Int 48 | 496.2(2.07mins |

### EXAMPLE 23

### 3-[({[(2S)-1-Methyl-2-pyrrolidinyl]methyl}oxy)methyl]-N-[(1S)-1-phenylpropyl]-2-(3-thienyl)-4-quinolinecarboxamide

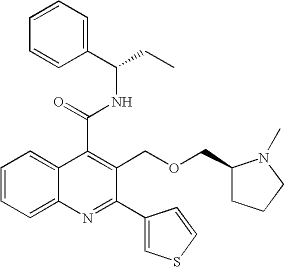

A stirred solution of Example 15 (120 mg, 0.25 mmole) in THF (6 ml) at room temperature under argon was treated with aqueous formaldehyde solution (0.12 ml of 37% w/v, 1.5 mmole) and sodium triacetoxyborohydride (110 mg, 0.50 mmole) and maintained for 1 hr. The solution was then concentrated under vacuum and the residue treated with 10% Na₂CO₃ solution and extracted with EtOAc. The extract was dried (Na₂SO₄), concentrated under vacuum and the residue chromatographed on silica gel eluting with 0-6% MeOH/DCM to afford the title compound as a colourless oil (90 mg, 73%). This was converted to its hydrochloride salt as a beige solid; ¹H NMR (400 MHz, CDCl₃) δ 1.03 (3H, t), 1.30-1.55 (1H, m), 1.60-1.90 (3H, m), 1.90-2.22 (4H, m), 2.27 (3H, s), 2.95-3.60 (3H, m), 4.40-4.65 (2H, m), 5.29 (1H, q), 6.92 (1H, br d), 7.27-7.45 (6H, m), 7.45-7.60 (2H, m), 7.72 (1H, t), 7.80-7.95 (2H, m), 8.10 (1H, d); m/z (APCI): 500.25 [M+H]⁺. Retention time 2.20 mins.

The following compounds of formula (Id) (see Table 8) were prepared by methods similar to that of Example 23 from the starting material indicated.

TABLE 8

| | |
|---|---|
| | (Id) |
| 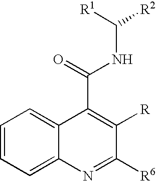 | |

| | | | | | | starting | m/z |
|---|---|---|---|---|---|---|---|
| | | | | | Starting | aldehyde/ | (retention |
| Ex | R¹ | R² | R⁶ | R | amine | ketone | time) |
| 24 | phenyl | ethyl | phenyl | 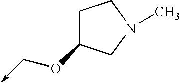 | Ex 16 | form-aldehyde | 480.3(2.37mins) |
| 25 | phenyl | ethyl | phenyl | 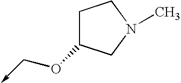 | Ex 17 | form-aldehyde | 480.3(2.37mins) |
| 26 | phenyl | ethyl | 2-thienyl | 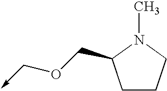 | Ex 14 | form-aldehyde | 500.4(2.25mins) |
| 27 | phenyl | ethyl | phenyl | 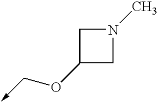 | Ex 18 | form-aldehyde | 466.3(2.35mins) |
| 28 | phenyl | ethyl | phenyl | 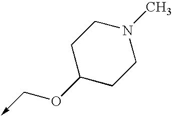 | Ex 19 | form-aldehyde | 494.3(2.37mins) |
| 29 | phenyl | ethyl | 3-F-phenyl | 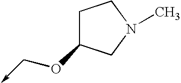 | Ex 20 | form-aldehyde | 498.3(2.42mins) |
| 30 | 3-F-phenyl | cyclo-propyl | phenyl | 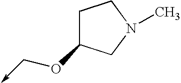 | Ex 22 | form-aldehyde | 510.4(2.10mins) |
| 31 | 3-F-phenyl | cyclo-propyl | 2-thienyl | 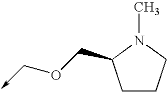 | Ex 21 | form-aldehyde | 530.3(2.28mins) |
| 32 | phenyl | ethyl | phenyl | 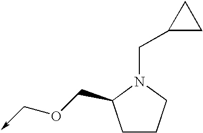 | Ex 12 | cyclopropylaldehyde | 534.3(2.62mins) |
| 33 | phenyl | ethyl | phenyl | 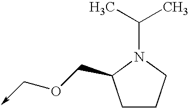 | Ex 12 | 2-propanone | 522.3(2.60mins) |
| 34 | phenyl | ethyl | phenyl | 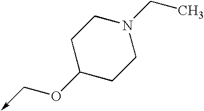 | Ex 12 | acetaldehyde | 508.3(2.40mins) |
| 35 | phenyl | ethyl | phenyl | 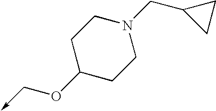 | Ex 12 | cyclopropylaldehyde | 534.4(2.49mins) |
| 36 | phenyl | ethyl | phenyl | 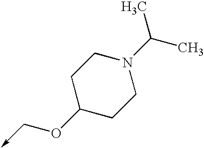 | Ex 12 | 2-propanone | 522.3(2.45mins) |
| 37 | phenyl | ethyl | phenyl | 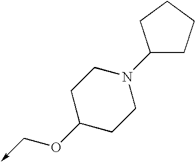 | Ex 12 | cyclo-pentanone | 548.3(2.52mins) |
| 38 | phenyl | isopropyl | 3-F-phenyl | 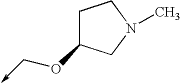 | Ex 20 | form-aldehyde | 512.3(2.48mins) |
| 39 | 3-F-phenyl | ethyl | phenyl | 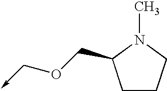 | Ex 13 | form-aldehyde | 512.3(2.49mins) |
| 40 | 3-F-phenyl | ethyl | phenyl | 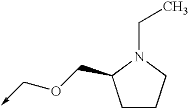 | Ex 13 | acetaldehyde | 526.3(2.52mins) |

### EXAMPLE 41

### 3-{[({(2S)-1-[2-(Methyloxy)ethyl]-2-pyrrolidinyl}methyl)oxy]methyl}-2-phenyl-N-[(1S)-1-phenylpropyl]-4-quinolinecarboxamide

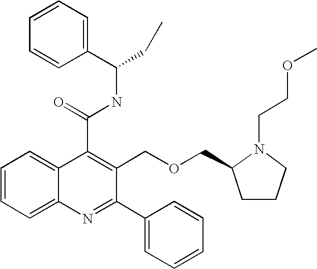

A stirred solution of Example 12 (150 mg, 0.31 mmole) in EtOH (5 ml) under argon was treated with 2-methoxyethyl bromide (55 mg, 0.40 mmole) and potassium carbonate (86 mg, 0.62 mmole) and heated at reflux for 20 hrs. Further 2-methoxyethyl bromide (55 mg, 0.40 mmole) was added and heating continued for 8 hrs, followed by more bromide (20 mg) and heating for an additional 8 hrs. The mixture was concentrated under vacuum and the residue treated with 10% Na₂CO₃ solution and extracted with EtOAc. The extract was dried (Na₂SO₄), concentrated under vacuum and the residual oil chromatographed on silica gel eluting with 0-3% MeOH/DCM to afford the title compound as a pale yellow oil (77 mg, 46%). This was converted to its hydrochloride salt as a beige solid. ¹H NMR (400 MHz, CDCl₃) δ 1.02 (3H, t), 1.40 (1H, br s), 1.62-1.83 (3H, m), 1.91-2.22 (3H, m), 2.30-2.45 (2H, m), 2.90 (1H, m), 3.00 (1H, br s), 3.06-3.13 (1H, m), 3.17 (3H, s), 3.22-3.40 (3H, m), 4.35-4.50 (2H, m), 5.28 (1H, q), 7.10 (1H, br s), 7.24-7.54 (9H, m), 7.62-7.75 (3H, m), 7.88 (1H, br s), 8.14 (1H, d); m/z 538.3 [M+H]⁺. Retention time 2.54 mins.

The following compounds of formula (Ie) (see Table 9) were prepared by similar methods to that of Example 41 from the starting material indicated.

TABLE 9

| | |
|---|---|
| | (Ie) |
| 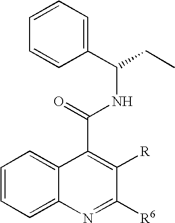 | |

| | | | | m/z |
|---|---|---|---|---|
| | | | Starting | (retention |
| Ex | R⁶ | R | material | time |
| 42 | phenyl | 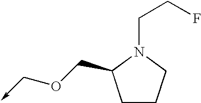 | 1-fluoro-2-iodoethane | 526.3(2.52 mins) |
| 43 | phenyl | 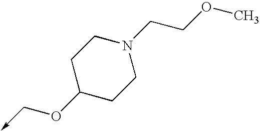 | 2-methoxy-ethylbromide | 538.3(2.42 mins) |
| 44 | 3-F-phenyl | 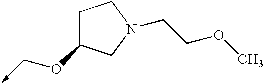 | 2-methoxy-ethylbromide | 542.3(2.48 mins) |

### Measurement of NK₃ Binding Affinity

The NK₃ binding affinity of the compounds of the invention was determined using the following scintillation proximity assay (SPA) (see H. M. Sarau et al, J. Pharmacol. Experimental Therapeutics 1997, 281(3), 1303-1311; H. M. Sarau et al, J. Pharmacol. Experimental Therapeutics 2000, 295(1), 373-381; G. A. M. Giardina et al J. Med. Chem. 1999, 42, 1053-1065). Polystyrene Leadseeker WGA-SPA beads (Amersham Biosciences) were mixed with plasma membrane prepared from CHO cell lines expressing NK₃ receptors in a bead/membrane ratio of 20:1 (w/w) in assay buffer (75 mM Tris pH 7.8, 75 mM NaCl, 4 mM MnCl₂, 1 mM EDTA, 0.05% Chaps, 1 mM PMSF). The mixture was placed on ice for 20 minutes to allow the formation of membrane/bead complex before BSA was added to a final concentration of 1%. After another 20 minutes incubation on ice, the bead/membrane complex was washed twice and suspended in assay buffer. ¹²⁵I [MePhe7]-NKB was then added to the bead/membrane complex. 10 μl of the resulting mixture was then dispensed into each well of a low volume Greiner 384-well plate with 100 nl compound pre-dispensed in 100% DMSO. The plates were then sealed and pulse spun at 1100 rpm. After 2-3 hours incubation at room temperature with shaking, the plates were spun for 2 min at 1100 rpm and measured in Viewlux imager (PerkinElmer) for 5 minutes with a 618-nm filter. Inhibition of the radioactive ligand binding to the NK₃ receptor was measured by the reduction of signal. pKᵢ was calculated using K_{d} of the radioactive ligand determined in a separate experiment.

The compounds of the invention antagonize the NK₃ receptor. The NK₃ binding affinity for all examples was determined using the above assay. All examples gave a pKᵢ equal to or greater than 7.5. The preferred compounds gave a pKᵢ equal to or greater than 8.2. Example 1 gave a pKᵢ of 8.2.

The therapeutic potential of the compounds of the invention can be assessed by measurement of the reversal of NK₃ agonist driven behaviours (e.g. contralateral turning in gerbils as described in Life Sciences 1995, 56, PL27-PL32 and Can. J. Physiol. Pharmacol. 2002, 80, 482-488; or guinea pig wet dog shakes as described in Br. J. Pharmacol. 1997, 122, 715-725) or by mechanistic correlates (e.g. electrophysiology of the dopamine cell firing as described in Gueudet et al., Synapse, 1999, 33, 71-79).

1. A compound of formula (I), a pharmaceutically acceptable salt, solvate or prodrug thereof 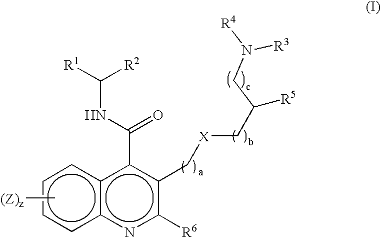
wherein
R¹ is phenyl optionally substituted by 1, 2 or 3 halogen atoms which halogen atoms may be the same or different;
R² is C₁₋₆alkyl, C₃₋₆cycloalkyl or acetyl;
X is oxygen or sulphur;
a is 1, 2 or 3;
b is 0 or 1;
c is 0, 1 or 2;
R³ is hydrogen or C₁₋₆alkyl;
R⁴ is hydrogen, C₁₋₆alkyl, haloC₁₋₆alkyl, C₁₋₄alkoxyC₁₋₆alkyl, C₃₋₆cycloalkyl or C₃₋₆cycloalkylC₁₋₆alkyl;
R⁵ is hydrogen; or R⁵ and R³, together with the interconnecting atoms, form a 4, 5 or 6 membered ring;
R⁶ is phenyl or thienyl, either of which is optionally substituted by 1, 2 or 3 halogen atoms, which atoms may be the same or different; and
z is 0, 1 or 2; wherein when z is 1 or 2, Z is a halogen atom, and wherein when z is 2 the halogen atoms may be the same or different.

2. A compound according to claim 1, a pharmaceutically acceptable salt, solvate or prodrug thereof, wherein R² is C₁₋₆alkyl or C₃₋₆cycloalkyl.

3. A compound according to claim 1, a pharmaceutically acceptable salt, solvate or prodrug thereof, wherein X is oxygen.

4. A compound according to claim 1, a pharmaceutically acceptable salt, solvate or prodrug thereof, wherein a is 1.

5. A compound according to claim 1, a pharmaceutically acceptable salt, solvate or prodrug thereof, wherein b is 1.

6. A compound according to claim 1, a pharmaceutically acceptable salt, solvate or prodrug thereof wherein R⁵ and R³, together with the interconnecting atoms, form a 4, 5 or 6 membered ring.

7. A compound according to claim 6, a pharmaceutically acceptable salt, solvate or prodrug thereof wherein b is 1, c is 0 and R⁵ and R³, together with the interconnecting atoms, form a 5 membered ring.

8. A compound according to claim 1, a pharmaceutically acceptable salt, solvate or prodrug thereof wherein R⁴ is C₁₋₆alkyl.

9. A compound according to claim 1, a pharmaceutically acceptable salt, solvate or prodrug thereof wherein R⁶ is phenyl.

10. A compound according to claim 1, a pharmaceutically acceptable salt, solvate or prodrug thereof wherein z is 0.

11. A compound according to claim 1, a pharmaceutically acceptable salt, solvate or prodrug thereof, having the formula (Ia):

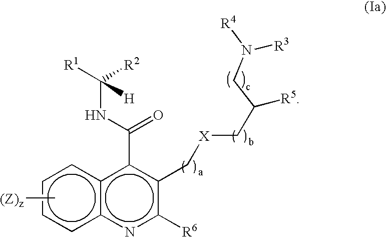

12. A compound according to claim 1, a pharmaceutically acceptable salt, solvate or prodrug thereof, wherein the compound is selected from the group consisting of:
3-[({[(2S)-1-Methyl-2-pyrrolidinyl]methyl}oxy)methyl]-2-phenyl-N-[(1S)-1-phenylpropyl]-4-quinolinecarboxamide;
N-[(1S)-2-Methyl-1-phenylpropyl]-3-[({[(2R)-1-methyl-2-pyrrolidinyl]methyl}oxy)methyl]-2-phenyl-4-quinolinecarboxamide;
3-({[2-(Dimethylamino)ethyl]thio}methyl)-2-phenyl-N-[(1S)-1-phenylpropyl]-4-quinolinecarboxamide;
3-({[2-(Diethylamino)ethyl]thio}methyl)-N-[(1S)-2-methyl-1-phenylpropyl]-2-phenyl-4-quinolinecarboxamide;
2-Phenyl-N-[(1S)-1-phenylpropyl]-3-({[(2S)-2-pyrrolidinylmethyl]oxy}methyl)-4-quinolinecarboxamide;
N-[(1S)-1-Phenylpropyl]-3-({[(2S)-2-pyrrolidinylmethyl]oxy}methyl)-2-(2-thienyl)-4-quinolinecarboxamide;
N-[(1S)-1-Phenylpropyl]-3-({[(2S)-2-pyrrolidinylmethyl]oxy}methyl)-2-(3-thienyl)-4-quinolinecarboxamide;
2-Phenyl-N-[(1S)-1-phenylpropyl]-3-{[(3S)-3-pyrrolidinyloxy]methyl}-4-quinolinecarboxamide;
2-Phenyl-N-[(1S)-1-phenylpropyl]-3-{[(3R)-3-pyrrolidinyloxy]methyl}-4-quinolinecarboxamide;
2-Phenyl-N-[(1S)-1-phenylpropyl]-3-[(4-piperidinyloxy)methyl]-4-quinolinecarboxamide;
2-(3-Fluorophenyl)-N-[(1S)-2-methyl-1-phenylpropyl]-3-{[(3S)-3-pyrrolidinyloxy]methyl}-4-quinolinecarboxamide;
N—[(S)-Cyclopropyl(3-fluorophenyl)methyl]-3-({[(2S)-2-pyrrolidinylmethyl]oxy}methyl)-2-(2-thienyl)-4-quinolinecarboxamide;
3-[({[(2S)-1-Methyl-2-pyrrolidinyl]methyl}oxy)methyl]-N-[(1S)-1-phenylpropyl]-2-(2-thienyl)-4-quinolinecarboxamide;
N—[(S)-Cyclopropyl(3-fluorophenyl)methyl]-3-[({[(2S)-1-methyl-2-pyrrolidinyl]methyl}oxy)methyl]-2-(2-thienyl)-4-quinolinecarboxamide;
3-[({[(2S)-1-(Cyclopropylmethyl)-2-pyrrolidinyl]methyl}oxy)methyl]-2-phenyl-N-[(1S)-1-phenylpropyl]-4-quinolinecarboxamide;
3-[({[(2S)-1-(1-Methylethyl)-2-pyrrolidinyl]methyl}oxy)methyl]-2-phenyl-N-[(1S)-1-phenylpropyl]-4-quinolinecarboxamide;
3-{[(1-Ethyl-4-piperidinyl)oxy]methyl}-2-phenyl-N-[(1S)-1-phenylpropyl]-4-quinolinecarboxamide;
3-({[1-(Cyclopropylmethyl)-4-piperidinyl]oxy}methyl)-2-phenyl-N-[(1S)-1-phenylpropyl]-4-quinolinecarboxamide;
3-({[1-(1-Methylethyl)-4-piperidinyl]oxy}methyl)-2-phenyl-N-[(1S)-1-phenylpropyl]-4-quinolinecarboxamide;
3-{[(1-Cyclopentyl-4-piperidinyl)oxy]methyl}-2-phenyl-N-[(1S)-1-phenylpropyl]-4-quinolinecarboxamide;
3-{[({(2S)-1-[2-(Methyloxy)ethyl]-2-pyrrolidinyl}methyl)oxy]methyl}-2-phenyl-N-[(1S)-1-phenylpropyl]-4-quinolinecarboxamide;
3-[({[(2S)-1-(2-Fluoroethyl)-2-pyrrolidinyl]methyl}oxy)methyl]-2-phenyl-N-[(1S)-1-phenylpropyl]-4-quinolinecarboxamide;
3-[({1-[2-(Methyloxy)ethyl]-4-piperidinyl}oxy)methyl]-2-phenyl-N-[(1S)-1-phenylpropyl]-4-quinolinecarboxamide;
and pharmaceutically acceptable salts, solvates and prodrugs thereof.

13. 3-[({[(2S)-1-Methyl-2-pyrrolidinyl]methyl}oxy)methyl]-2-phenyl-N-[(1S)-1-phenylpropyl]-4-quinolinecarboxamide.

14-18. (canceled)

19. A method of treatment of a disease or condition mediated by modulation of the NK3 receptor in a mammal comprising administering an effective amount of a compound as claimed in claim 1.

20. A method as claimed in claim 19 wherein the disease or condition is depression; anxiety disorder; phobia; psychosis or a psychotic disorder.

21. A pharmaceutical composition comprising a compound as claimed in claim 1 and one or more pharmaceutically acceptable carrier(s), diluents(s) and/or excipient(s).
